(19) United States
(12) Patent Application Publication
(10) Pub. No.: US 2007/0129562 A1
(43) Pub. Date: Jun. 7, 2007

(54) Process for the preparation of highly pure 1-[2- dimethylamino-(4-methoxyphenyl)ethyl]cyclohexanol hydrochloride

(76) Inventors: Vinod Kumar Kansal, Haryana (IN); Brijnath P. Chaurasia, Uttar Pradesh (IN); Mahagare Sachin Jaysing, Maharashtra (IN); Shivaji Haribhau Shelke, Maharashtra (IN)

Correspondence Address: KENYON & KENYON LLP, ONE BROADWAY, NEW YORK, NY, 10004, US

(21) Appl. No.: 11/584,716

(22) Filed: Oct. 19, 2006

Related U.S. Application Data
(60) Provisional application No. 60/728,632, filed on Oct. 19, 2005.
(60) Provisional application No. 60/789,086, filed on Apr. 3, 2006.
(60) Provisional application No. 60/798,877, filed on May 8, 2006.

(51) Int. Cl. C07C 253/30

(52) U.S. Cl. 558/351

(57) ABSTRACT

The invention encompasses processes for the preparation of highly pure venlafaxine hydrochloride, 1-[2-dimethylamino-(4-methoxyphenyl)ethyl]cyclohexanol hydrochloride, as well as intermediates thereof.

### CROSS-REFERENCE TO RELATED APPLICATIONS

This application claims the benefit of priority to U.S. provisional application Ser. Nos. 60/728,632, filed Oct. 19, 2005, 60/789,086, filed Apr. 3, 2006, and 60/798,877, filed May 8, 2006, hereby incorporated by reference.

### FIELD OF THE INVENTION

The invention encompasses processes for the preparation of highly pure venlafaxine hydrochloride, 1-[2-dimethylamino-(4-methoxyphenyl)ethyl]cyclohexanol hydrochloride, as well as intermediates thereof.

### BACKGROUND OF THE INVENTION

Venlafaxine, 1-[2-dimethylamino-(4-methoxyphenyl)ethyl]-cyclohexanol, a compound having the chemical formula (I) 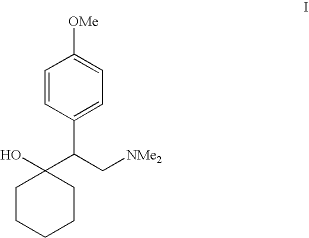
and its pharmaceutically acceptable salts are important antidepressants of the central nervous system. Venlafaxine acts by inhibiting re-uptake of norepinephrine and serotonin selective re-uptake inhibitors.

The preparation of venlafaxine has been disclosed in several patents. For example, U.S. Pat. No. 4,535,186 to Husbands et al., describes the preparation of the compound of Formula (I) by reacting p-Methoxyphenyl acetonitrile of Formula (V) 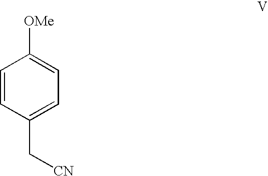
in the presence of n-butyl lithium at a temperature of −50 to −70° C., with cyclohexanone of Formula (VI) 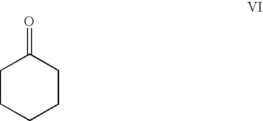
to form the compound of Formula (II) (See Sauvetre, et al., “Factors controlling the regioselectivity of additions to α-enones-VI: Reactions of acetonitrile and phenylacetonitrile anions,” Tetrahedron, 34(14), 2135-2140 (1978)), 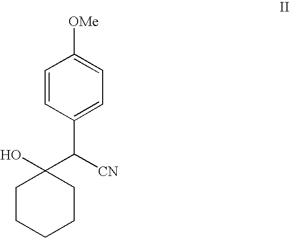
followed by reduction under hydrogen pressure with rhodium on alumina catalyst, to form the compound of Formula (III). 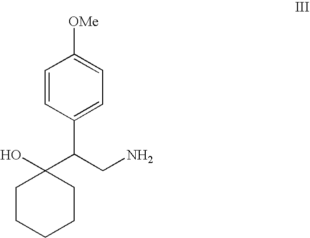
The compound is then N-methylated employing formalin, formic acid and water as described by Tilford, et al. See Tilford, et al., “Diuretics. α,α-Disubstituted 2-Piperidine-ethanols and 3,3-Disubstituted Octahydropyrid[1,2-c]oxazines,” J. Am. Chem. Soc., 76(9), 2431-2441 (1954). Alternatively, the reduction may be carried out using the procedure described by Borch and Hassid for N-methylation, using sodium cyanoborohydride and formaldehyde. See J. Org. Chem., 37, 1663 (1972).

The process disclosed in U.S. Pat. No. 4,535,186 uses n-butyl lithium, a reportedly inflammable and pyrophoric substance, at low temperatures, e.g., −50 to −70° C., in the course of reaction to obtain the compound of Formula (II).

PCT publication No. WO 00/32556, describes a process for the preparation of the compound of Formula (II) by the condensation of p-methoxyphenyl acetonitrile of Formula (V) with cyclohexanone of Formula (VI) in the presence of lithium diisopropylamide, a reportedly corrosive and unstable substance, at temperatures in the range of −65° C. to −78° C. As with the process disclosed in U.S. Pat. No. 4,535,186, low temperatures and reportedly hazardous reagents make this process commercially unattractive.

Chinese patent No. 1225356 describes a process for the preparation of the compound of Formula (II) by the condensation of p-methoxyphenyl acetonitrile of Formula (V) with cyclohexanone of Formula (VI) in the presence of sodium methoxide, sodium ethoxide, sodium amide, or sodium hydride in cyclohexane at temperatures in the range of 0° C. to 5° C. The compound of Formula (II) is then reduced to the compound of Formula (III) with sodium borohydride and boron trifluoride diethyl ether complex at reflux temperature.

Chinese patent No. 1225356 uses sodium borohydride and boron trifluoride ethyl ether as the reducing agent for the reduction of compound of Formula (II) to the compound of Formula (III) at reflux resulting in the yield of 81%.

PCT publication No. WO 02/18325, filed by Ciba Specialty Chemicals, discloses a process for the preparation of the compound of Formula (II) by reacting 4-methoxyphenylacetonitrile with cyclohexanone in the presence of an aqueous base and a phase transfer catalyst at 0 to 60° C.

PCT publication No. WO 02/50017 discloses the reduction of the compound of Formula (II) in the presence of a nickel or cobalt catalyst.

U.S. Pat. No. 6,350,912, discloses the one-pot preparation of venlafaxine by reduction of the compound of Formula (II) in the presence of Raney nickel to form the compound of Formula (III), followed by conversion to venlafaxine in yields of 15-28%.

Indian patent application No. IN 194085 discloses a method for the preparation of venlafaxine of Formula (I) by combining p-methoxyphenyl acetonitrile of Formula (V) with cyclohexanone of Formula (VI) and sodium hydroxide in an alcoholic solvent to produce the compound of Formula (II), and reducing the compound of Formula (II) with NaBH₄ in presence of carboxylic acid in an aprotic solvent to prepare the compound of Formula (III).

In U.S. publication No. 2005/0033088, the compound of Formula (II) was reduced in the presence of palladium on charcoal catalyst in the presence of an organic acid selected from formic acid, acetic acid or propionic acid. The yield of the obtained product of Formula (III) is reported as 45-55%.

Many of the above processes have features that make them less than optimal for use on an industrial scale. Hence, there is a need in the art for processes for the preparation of venlafaxine that are suitable for use on an industrial scale.

### SUMMARY OF THE INVENTION

The invention encompasses processes for the preparation of venlafaxine of Formula (I) and intermediates thereof according to Scheme 1.

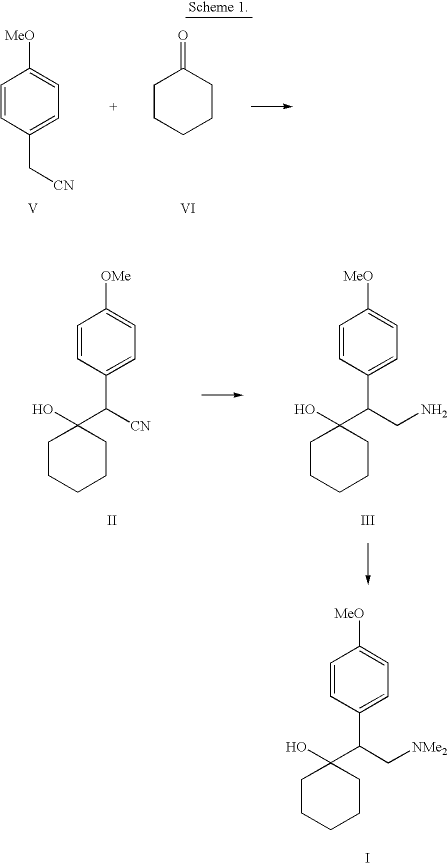

In one embodiment, the invention encompasses a process for preparing 1-[cyano-(4-methoxyphenyl)methyl]-cyclohexanol of Formula (II) comprising: reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II); and recovering the 1-[cyano-(4-methoxyphenyl)methyl]-cyclohexanol of Formula (II).

In another embodiment, the invention encompasses a process for preparing venlafaxine of Formula (I) or a pharmaceutically acceptable salt thereof comprising: reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II); converting the 1-[cyano-(4-methoxyphenyl)methyl]-cyclohexanol of Formula (II) into 1-[2-amino-1-(4-methoxyphenyl)ethyl]-cyclohexanol of Formula (III); converting the 1-[2-amino-1-(4-methoxyphenyl)ethyl]-cyclohexanol of Formula (III) into venlafaxine of Formula (I); and optionally converting the venlafaxine of Formula (I) into a pharmaceutically acceptable salt thereof.

In another embodiment, the invention encompasses a process for preparing 1-[2-amino-1-(4-methoxyphenyl)ethyl]-cyclohexanol of Formula (III) comprising: reacting 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); and recovering the 1-[2-amino-1-(4-methoxyphenyl)ethyl]-cyclohexanol of Formula (III).

In another embodiment, the invention encompasses a process for preparing 1-[2-amino-1-(4-methoxyphenyl)ethyl]-cyclohexanol of Formula (III) comprising: reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II); reacting the 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); and recovering the 1-[2-amino-1-(4-methoxyphenyl)ethyl]-cyclohexanol of Formula (III).

In another embodiment, the invention encompasses a process for preparing 1-[2-amino-1-(4-methoxyphenyl)ethyl]-cyclohexanol hydrochloride of Formula (III-a) comprising: combining 1-[2-amino-1-(4-methoxyphenyl)ethyl]-cyclohexanol of Formula (III), a water immiscible solvent and hydrochloric acid to form a mixture; and recovering 1-[2-amino-1-(4-methoxyphenyl)ethyl]-cyclohexanol hydrochloride of Formula (III-a) from the mixture. Preferably, the 1-[2-amino-1-(4-methoxyphenyl)ethyl]-cyclohexanol hydrochloride of Formula (III-a) is recovered in a purity of greater than about 98% area by HPLC, and more preferably in a purity of about 99.9% area by HPLC.

In another embodiment, the invention encompasses a process for preparing 1-[2-amino-1-(4-methoxyphenyl)ethyl]-cyclohexanol of Formula (III-a) comprising: reducing 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); combining the 1-[2-amino-1-(4-methoxyphenyl)ethyl]-cyclohexanol of Formula (III), a water immiscible solvent and hydrochloric acid to form a mixture; and recovering 1-[2-amino-1-(4-methoxyphenyl)ethyl]-cyclohexanol hydrochloride of Formula (III-a) from the mixture.

In another embodiment, the invention encompasses a process for preparing venlafaxine of Formula (I) or a pharmaceutically acceptable salt thereof comprising: combining 1-[2-amino-1-(4-methoxyphenyl)ethyl]-cyclohexanol of Formula (III) with formaldehyde, formic acid, and water to form a reaction mixture; heating the reaction mixture at reflux for a period of time sufficient to carry out N-methylation of the 1-[2-amino-1-(4-methoxyphenyl)ethyl]-cyclohexanol of Formula (III); cooling the reaction mixture to a temperature of about 25° C. to about 20° C.; adding a base in an amount sufficient to adjust the pH of the reaction mixture to about 9.5 to about 11.5; extracting the aqueous phase with a water immiscible organic solvent to obtain a precipitate of venlafaxine of Formula (I); recovering the precipitated venlafaxine of Formula (I); and optionally converting the venlafaxine of Formula (I) into a pharmaceutically acceptable salt thereof.

In another embodiment, the invention encompasses a process for preparing venlafaxine of Formula (I) or a pharmaceutically acceptable salt thereof comprising: reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II); reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III); combining 1-[2-amino-1-(4-methoxyphenyl)ethyl]-cyclohexanol of Formula (III) with formaldehyde, formic acid, and water to form a reaction mixture; heating the reaction mixture at reflux for a period of time sufficient to carry out N-methylation of the 1-[2-amino-1-(4-methoxyphenyl)ethyl]-cyclohexanol of Formula (III); cooling the reaction mixture to a temperature of about 25° C. to about 20° C.; adding a base in an amount sufficient to adjust the pH of the reaction mixture to about 9.5 to about 11.5; extracting the aqueous phase with a water immiscible organic solvent to obtain a precipitate of venlafaxine of Formula (I); recovering the precipitated venlafaxine of Formula (I); and optionally converting the venlafaxine of Formula (I) into a pharmaceutically acceptable salt thereof.

In another embodiment, the invention encompasses a one-pot process for preparing venlafaxine of Formula (I) or a pharmaceutically acceptable salt thereof comprising: providing 1-[cyano-(4-methoxyphenyl)methyl]-cyclohexanol of Formula (II); reducing the 1-[cyano-(4-methoxyphenyl)methyl]-cyclohexanol of Formula (II) with sodium borohydride and boron trifluoride ethyl ether in at least one aprotic solvent to obtain 1-[2-amino-1-(4-methoxyphenyl)ethyl]-cyclohexanol of Formula (III) in situ; quenching the resulting reaction mass; N-methylating the 1-[2-amino-1-(4-methoxyphenyl)ethyl]-cyclohexanol of Formula (III) with formaldehyde and formic acid to form venlafaxine of Formula (I); and optionally converting the venlafaxine of Formula (I) into a pharmaceutically acceptable salt thereof. Preferably, the pharmaceutically acceptable salt is venlafaxine hydrochloride of Formula (IV). Preferably, the venlafaxine hydrochloride of Formula (IV) is obtained in a purity of greater than about 98% area by HPLC, more preferably in a purity of greater than about 99% area by HPLC, even more preferably in a purity of greater than about 99.5% area by HPLC, and most preferably in a purity of greater than about 99.8% area by HPLC.

In another embodiment, the invention encompasses a one-pot process for preparing venlafaxine of Formula (I) or a pharmaceutically acceptable salt thereof comprising: providing 1-[cyano-(4-methoxyphenyl)methyl]-cyclohexanol of Formula (II); reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III); N-methylating the 1-2[amino-1-(4-methoxyphenyl)ethyl]-cyclohexanol of Formula (III) with formaldehyde and formic acid to form venlafaxine of Formula (I); recovering the venlafaxine of Formula (I) from the reaction mixture; and optionally converting the venlafaxine of Formula (I) into a pharmaceutically acceptable salt thereof. Preferably, the venlafaxine hydrochloride of Formula (IV) is obtained in a purity of greater than about 98% area by HPLC, more preferably in a purity of greater than about 99% area by HPLC, even more preferably in a purity of greater than about 99.5% area by HPLC, and most preferably in a purity of greater than about 99.8% area by HPLC.

In another embodiment, the invention encompasses a process for preparing venlafaxine of Formula (I) or a pharmaceutically acceptable salt thereof comprising: reacting p-methoxyphenyl acetonitrile of Formula (V) with cyclohexanone of Formula (VI) in the presence of at least one alkali alkoxide in at least one of methanol and ethanol at a temperature of about 10° C. to about 25° C. to form 1-[cyano-(4-methoxyphenyl)methyl]-cyclohexanol of Formula (II); reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III); N-methylating the 1-[2-amino-1-(4-methoxyphenyl)ethyl]-cyclohexanol of Formula (III) with formaldehyde and formic acid to form venlafaxine of Formula (i); and optionally converting the venlafaxine of Formula (I) into a pharmaceutically acceptable salt thereof.

### DETAILED DESCRIPTION OF THE INVENTION

The invention encompasses processes for preparing venlafaxine and intermediates thereof that are suitable for use on an industrial scale, according to Scheme 1. Scheme 1.

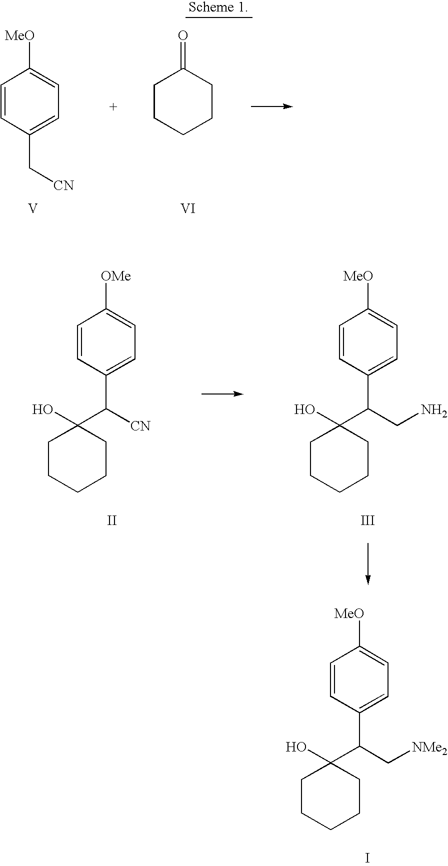

In one embodiment, the invention encompasses a process for preparing the intermediate 1-[cyano-(4-methoxyphenyl)methyl]-cyclohexanol of Formula (II) comprising reacting p-methoxyphenyl acetonitrile of Formula (V) with cyclohexanone of Formula (VI) in the presence of at least one alkali alkoxide in at least one of methanol and ethanol to form the intermediate 1-[cyano-(4-methoxyphenyl)methyl]-cyclohexanol of Formula (II); and recovering the intermediate 1-[cyano-(4-methoxyphenyl)methyl]-cyclohexanol of Formula (II).

Typically, the alkali alkoxide is based on an alkoxide having from 1 to 6 carbon atoms. Preferably, the alkali alkoxide is sodium ethoxide, sodium methoxide, potassium ethoxide, potassium methoxide, or potassium tert-butoxide. Preferably, the alkali alkoxide is sodium ethoxide or sodium methoxide. These alkali alkoxides are desirable because they are commercially available and inexpensive.

Typically, the reaction is performed at a temperature of about −50° C. to about 15° C. Preferably, the reaction is performed at a temperature of about −10° C. to about 15° C. More preferably, the reaction is performed at a temperature of about 0° C. to about 5° C.

Typically, the p-methoxyphenyl acetonitrile of Formula (V), alkali alkoxide, and cyclohexanone of Formula (VI) are present in a molar ratio of about 1:1-2:1-3 or less, i.e., one mole equivalent of p-methoxyphenyl acetonitrile of Formula (V), one to two mole equivalent of alkali alkoxide and one to three mole equivalent of cyclohexanone of Formula (VI). Preferably, the p-methoxyphenyl acetonitrile of Formula (V), alkali alkoxide, and cyclohexanone of Formula (VI) are combined in a molar ratio of about 1:1.2-1.5:1.5-2, i.e., one equivalent of p-methoxyphenyl acetonitrile of Formula (V), one point two to one point five mole equivalent of alkali alkoxide and one point five to two mole equivalent of cyclohexanone of Formula (VI).

Typically, the intermediate 1-[cyano-(4-methoxyphenyl)methyl]-cyclohexanol of Formula (II) is produced from the p-methoxyphenyl acetonitrile of Formula (V) and cyclohexanone of Formula (VI) in at least about 98% yield.

The intermediate 1-[cyano-(4-methoxyphenyl)methyl]-cyclohexanol of Formula (II) thus prepared can be reduced to the intermediate 1-[2-amino-1-(4-methoxyphenyl)ethyl]-cyclohexanol of Formula (III) by conventional techniques, which can then be converted into venlafaxine.

The invention also encompasses a process for preparing the intermediate 1-[2-amino-1-(4-methoxyphenyl)ethyl]-cyclohexanol of Formula (III) comprising reacting 1-[cyano-(4-methoxyphenyl)methyl]-cyclohexanol of Formula (II) with hydrogen and a hydrogenation catalyst in the presence of at least one inorganic acid in at least one alcohol; and recovering the intermediate 1-[2-amino-1-(4-methoxyphenyl)ethyl]-cyclohexanol of Formula (III).

The starting 1-[cyano-(4-methoxyphenyl)methyl]-cyclohexanol of Formula (II) can be obtained by the above-described process, or by any method known to one of ordinary skill in the art.

Typically, the alcohol is a C₁-C₆ alcohol. Preferably, the alcohol is methanol, ethanol, isopropanol, n-propanol, n-butanol, isobutanol, or t-butanol. More preferably, the alcohol is methanol, ethanol, or isopropanol.

Typically, the hydrogenation catalyst is palladium on charcoal, platinum on charcoal, rhodium on charcoal, or rhodium on alumina.

Typically, the inorganic acid is hydrochloric acid, hydrochloric acid from acetyl chloride, ammonium chloride, hydrobromic acid, hydrobromic acid from acetyl bromide, or ammonium bromide. Preferably, the inorganic acid is hydrochloric acid.

Typically, the inorganic acid is present in a molar ratio of 0.5 to 5 mole equivalent per mole of the compound of Formula (II). Preferably, the inorganic acid is present in a molar ratio of 1 to 3 mole equivalent per mole of the compound of Formula (II).

Typically, the hydrogen is applied at a pressure of about 5 to about 25 kg/cm². Preferably, the hydrogen pressure applied is about 10 to about 20 kg/cm² and more preferably about 15 to about 20 kg/cm².

Typically, the temperature maintained during hydrogenation is about 25° C. to about 65° C. Preferably, the temperature maintained during hydrogenation is about 35° C. to about 60° C. and more preferably about 40° C. to about 50° C.

The intermediate 1-[2-amino-1-(4-methoxyphenyl)ethyl]-cyclohexanol of Formula (III) can optionally be converted into its hydrochloride salt, 1-[2-amino-1-(4-methoxyphenyl)ethyl]-cyclohexanol hydrochloride of Formula (III-a) by reaction with hydrochloric acid.

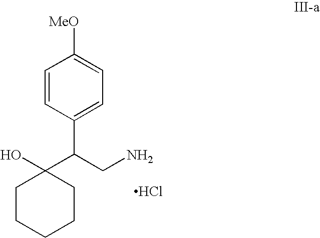

Preferably, the process comprises: combining the intermediate 1-[2-amino-1-(4-methoxyphenyl)ethyl]-cyclohexanol of Formula (III), a water immiscible solvent and hydrochloric acid to form a mixture; and recovering the 1-[2-amino-1-(4-methoxyphenyl)ethyl]-cyclohexanol hydrochloride of Formula (III-a) from the mixture.

Typically, the water immiscible solvent is at least one of ethyl acetate, n-heptane, toluene, or methylene dichloride.

Typically, the hydrochloric acid is in the form of a solution, such as in isopropyl alcohol.

Typically, the compound of Formula (III-a) is obtained from the compound of Formula (III) in about 70-80% yield. Typically, the compound of Formula (III-a) is obtained with a purity of greater than about 98% area by HPLC and preferably with a purity of about 99.9%.

The intermediate 1-[2-amino-1-(4-methoxyphenyl)ethyl]-cyclohexanol of Formula (III) or its hydrochloride salt (III-a) thus prepared can be converted to venlafaxine by conventional techniques.

Venlafaxine of Formula (I) may be prepared by a process comprising N-methylating the intermediate 1-[2-amino-1-(4-methoxyphenyl)ethyl]-cyclohexanol of Formula (III) with formaldehyde and formic acid.

Preferably, the process comprises: combining the intermediate 1-[2-amino-1-(4-methoxyphenyl)ethyl]-cyclohexanol of Formula (III) with formaldehyde, formic acid, and water to form a reaction mixture; heating the reaction mixture at reflux for a period of time sufficient to carry out N-methylation; cooling the reaction mixture, preferably, to a temperature of about 25° C. to about 20° C.; adding a base in an amount sufficient to adjust the pH of the reaction mixture, preferably, to about 9.5 to about 11.5; and extracting the aqueous phase with a water immiscible organic solvent to obtain a precipitate of 1-[2-dimethylamino-1-(4-methoxyphenyl)ethyl]-cyclohexanol of Formula (I).

The starting 1-[2-amino-1-(4-methoxyphenyl)ethyl]-cyclohexanol of Formula (III) can be obtained by the above-described process, or by any method known to one of ordinary skill in the art.

Preferably, the base is added in an amount sufficient to adjust the pH of the reaction mixture to about 9.5 to about 10.5.

Preferably, the water immiscible organic solvent is ethyl acetate, n-heptane, toluene, or methylene dichloride.

The venlafaxine of Formula (I) can optionally be converted into a pharmaceutically acceptable salt, including, but not limited to, the hydrochloride salt, 1-[2-dimethylamino-1-(4-methoxyphenyl)ethyl]-cyclohexanol hydrochloride, of Formula (IV).

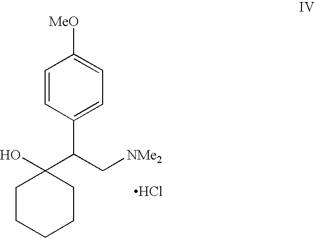

The process for preparing venlafaxine may be carried out in one pot, without recovering the intermediates. In one embodiment, venlafaxine of Formula (I) or a pharmaceutically acceptable salt thereof is prepared by providing 1-[cyano-(4-methoxyphenyl)methyl]-cyclohexanol of Formula (II); reacting the 1-[cyano-(4-methoxyphenyl)methyl]-cyclohexanol of Formula (II) with a reducing agent to form 1-[2-amino-1-(4-methoxyphenyl)ethyl]-cyclohexanol of Formula (III); N-methylating the 1-[2-amino-1-(4-methoxyphenyl)ethyl]-cyclohexanol of Formula (III) with formaldehyde and formic acid to form venlafaxine of Formula (I); and, optionally converting the venlafaxine of Formula (I) to a pharmaceutically acceptable salt.

Typically, the reducing agent is MBH₄ or hydrogen in the presence of hydrogenation catalyst, wherein M is a metal cation or a quaternary ammonium cation.

When the reducing agent is hydrogen in the presence of hydrogenation catalyst, the reaction conditions for the reducing step are as set forth above.

Preferably, the intermediate 1-[cyano-(4-methoxyphenyl)methyl]-cyclohexanol of Formula (II) is reduced with sodium borohydride and boron trifluoride, in the presence of at least one aprotic solvent.

Typically, about 2 to 4 mole equivalents of sodium borohydride and about 1 to 3 mole equivalents of boron trifluoride ethyl ether are combined per mole of 1-[cyano-(4-methoxyphenyl)methyl]-cyclohexanol of Formula (II). Preferably, the compound of Formula (II), sodium borohydride, and boron trifluoride ethyl ether are combined in a molar ratio of 1:4:3, more preferably, in a molar ratio of 1:3:2, and most preferably, in a molar ratio of 1:2:1.

Typically, the aprotic solvent is tetrahydrofuran, tert-butylmethyl ether, or di-isopropyl methyl ether. Preferably, the aprotic solvent is tetrahydrofuran.

Typically, the temperature during the reduction is about −10° C. to about 55° C. Preferably, the temperature during the reduction is about −10° C. to about 45° C., more preferably, about 0° C. to about 5° C. The temperature may be gradually increased during the progress of the reduction. Preferably, the initial temperature during reduction is −10° C. to 20° C., more preferably, −5° C. to 15° C., most preferably −5° C. to 10° C. During the progress of the reduction, the temperature is preferably increased to about 5° C. to 25° C., and then to about 25° C. to 45° C.

After reduction is complete, the reaction mixture is typically quenched before N-methylating the obtained 1-[2-amino-1-(4-methoxyphenyl)ethyl]-cyclohexanol of Formula (III). Preferably, the reaction mixture is quenched by the addition of at least one organic or inorganic acid.

Typically, the organic acid is formic acid, acetic acid, or adipic acid. Preferably, the organic acid is formic acid. Typically, the inorganic acid is HCl or H₂SO₄. Preferably, the inorganic acid is HCl.

Typically, the quenching is performed at a temperature of about 0° C. to about 5° C.

The other reaction conditions for the one-pot process are as set forth in the processes above.

In another embodiment, the invention encompasses a process for preparing venlafaxine of Formula (I) or a pharmaceutically acceptable salt thereof comprising: reacting p-methoxyphenyl acetonitrile of Formula (V) with cyclohexanone of Formula (VI) in the presence of at least one alkali alkoxide in at least one of methanol and ethanol at a temperature of about 10° C. to about 25° C. to form 1-[cyano-(4-methoxyphenyl)methyl]-cyclohexanol of Formula (II); reacting 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); N-methylating the 1-[2-amino-1-(4-methoxyphenyl)ethyl]-cyclohexanol of Formula (III) with formaldehyde and formic acid to form venlafaxine of Formula (I); and optionally converting the venlafaxine of Formula (I) into a pharmaceutically acceptable salt thereof.

The compound of Formula (IV) is produced by the above-described processes in a purity of greater than about 98% area by HPLC. Preferably, the compound of Formula (IV) is produced in a purity of greater than about 99% area by HPLC, more preferably greater than about 99.5%, and most preferably greater than about 99.8%.

The compound of Formula (IV) thus obtained can be formulated into a pharmaceutical composition by admixing the compound of Formula (IV) and at least one pharmaceutically acceptable excipient. Suitable excipients include, but are not limited to, diluents, carriers, fillers, bulking agents, binders, disintegrants, disintegration inhibitors, absorption accelerators, wetting agents, lubricants, glidants, surface active agents, flavoring agents, and the like. Selection of excipients and the amounts to use can be readily determined by an experienced formulation scientist in view of standard procedures and reference works known in the art.

The pharmaceutical composition can be formulated into a solid or a liquid dosage form for administration to a patient. Dosage forms include, but are not limited to, tablets, capsules, powders, syrups, suspensions, emulsions, injection preparations, and the like.

While the present invention is described with respect to particular examples and preferred embodiments, it is understood that the present invention is not limited to these examples and embodiments. The present invention as claimed therefore includes variations from the particular examples and preferred embodiments described herein, as will be apparent to one of skill in the art.

### EXAMPLES

HPLC Method for Measuring Chemical Purity of Venlafaxine:

A high performance liquid chromatograph with a Zorbax SB-C-18,250×4.6 mm column and an ultraviolet detector at 230 nm was used. The injection volume was 10 μl, the flow rate was 1 ml/minute, and the column temperature was 25° C.

The mobile phase was comprised of two eluents (A and B). Eluent A was 30% acetonitrile and 70% buffer. The buffer was prepared by adding 4.0 ml of trifluoroacetic acid and 7.0 ml of triethylamine to 1 liter of water, and adjusting the pH to 3.0 with triethylamine. Eluent B was prepared by adding 1.6 ml of trifluoroacetic acid and 2.9 ml of triethylamine to 700 ml acetonitrile and 300 ml buffer, and adjusting the pH of the solution to 3.0 with triethylamine or trifluoroacetic acid, if necessary.

Samples of venlafaxine prepared by the processes herein described were dissolved in acetonitrile, diluted with eluent A, and injected into the chromatograph. The samples were carried through the column by gradient elution under the following conditions: 55 minutes of 100% eluent A, followed by a mixture of 45% eluent A: 55% eluent B.

### Example 1

### Preparation of 1-[Cyano-(4-methoxyphenyl)methyl]-cyclohexanol (II)

A four-necked flask equipped with an addition funnel, thermometer pocket, drying tube and mechanical stirrer, was charged with methanol (300 ml) and sodium methoxide (55.05 g, 1.02 mole) at a temperature of 25° C. to 30° C. followed by addition of methanol (50 ml). The mixture was cooled to 0° C. to 5° C., followed by addition of 4-methoxyphenylacetonitrile (100 g, 0.679 mole) over a period of 15-20 minutes at a temperature of 0° C. to 5° C. The reaction mixture was additionally stirred for 60-70 minutes, at a temperature of 0° C. to 5° C. Cyclohexanone of Formula (VI) (116.69 g, 1.19 mole) was added slowly over a period of 60-70 minutes at a temperature of 0° C. to 5° C. and the mixture was stirred for 2.5-3.0 hrs at this temperature. The reaction mass was quenched with a solution of acetic acid (1.02 mole) in water (500 ml) and stirred for 25-30 minutes at a temperature of 0-5° C. Subsequently, the mass was filtered, crystallized from toluene, and then dried under vacuum at 45° C. to 50° C. to yield 142-150 g (85-90% yield) of a white solid 1-[Cyano-(4-methoxyphenyl)methyl]-cyclohexanol with a purity of 99.8% area by HPLC.

### Example 2

### Preparation of 1-[2-Dimethylamino-1-(4-methoxyphenyl)ethyl]-cyclohexanol hydrochloride (IV)

A four-necked flask equipped with an addition funnel, thermometer pocket, drying tube and mechanical stirrer, was charged with tetrahydrofuran (150 ml), sodium borohydride (35.46 g, 0.938 mole) at a temperature of 25° C. to 30° C. followed by addition of tetrahydrofuran (50 ml). The mixture was cooled to 0° C. to 5° C., followed by addition of boron trifluoride ethyl ether complex (115.7 g, 0.815 mole) diluted with tetrahydrofuran (100 ml) over a period of 60-90 minutes at a temperature of 0° C. to 5° C. A solution of 1-[cyano-(4-methoxyphenyl)methyl]-cyclohexanol (100 g, 0.408 mole) in tetrahydrofuran (200 ml) was added slowly over a period of 60-90 minutes at a temperature of 0° C. to 5° C. Then, the mixture was stirred for 2-2.5 hrs at a temperature of 20° C. to 25° C. followed by stirring at a temperature of 30° C. to 40° C. for an additional 2-2.5 hours. The reaction mixture was cooled to 20° C. to 25° C. and slowly quenched with an aqueous solution of formic acid (310 ml) containing 90% formic acid (40 ml) at a temperature of 10° C. to 15° C. and then was stirred for 30-40 minutes at 20° C. to 25° C. The organic solvents were distilled out at 45° C. to 95° C. followed by addition of formic acid (93.9 g, 1.8 mole), formaldehyde (33.82 g, 1.13 mole) and water (500 ml). The reaction mass was heated to reflux for 8-16 h and subsequently it was cooled to 20° C. to 25° C. The pH of the aqueous mass was adjusted to 9.5-11.5 by adding a solution of 50% sodium hydroxide. The aqueous phase was extracted with ethyl acetate (3×300 ml), followed by washing with water (200 ml) and saturated sodium chloride solution (200 ml) and drying the combined ethyl acetate extracts over anhydrous sodium sulphate. The solvent ethyl acetate (400 ml) was distilled out under vacuum at 45° C. to 50° C., to obtain free base solution. To the above free base solution, 16% isopropanol hydrochloric acid solution (110 g) was slowly added at a temperature of 20° C. to 45° C. followed by stirring at a temperature of 5° C. to 10° C. for 90-120 minutes. The solid separated was filtered, washed with ethyl acetate (100 ml) and then dried under vacuum at 35° C. to 45° C. to obtain 100-105 g (78.25-82.17% yield) of a white solid 1-[2-Dimethylamino-1-(4-methoxyphenyl)ethyl]-cyclohexanol hydrochloride with a purity 99.82% area by HPLC.

### Example 3

### Preparation of 1-[Cyano-(4-methoxyphenyl)methyl]-cyclohexanol (II)

A four-necked flask equipped with an addition funnel, thermometer pocket, drying tube and mechanical stirrer, was charged with methanol (300 ml), sodium methoxide (55.05 g, 1.02 mole) at a temperature of 25-30° C. followed by addition of methanol (50 ml). The mixture was cooled to 0-5° C., followed by addition of 4-methoxyphenylacetonitrile (100 g, 0.679 mole) over a period of 15-20 minutes at a temperature of 0-5° C. The reaction mixture was stirred for an additional 60-70 minutes, at a temperature of 0-5° C. Cyclohexanone of Formula (VI) (116.69 g, 1.19 mole) was added slowly over a period of 60-70 minutes at a temperature of 0-5° C. and the mixtures was stirred for 2.5-3.0 hrs at this temperature. The reaction mass was quenched with chilled (3-5° C.) water (500 ml) and stirred for 25-30 minutes at a temperature of 0-5° C. and filtered, washed with water and methanol and then dried under vacuum at 35-40° C. to yield 145.03 g (87% yield) of a white solid 1-[Cyano-(4-methoxyphenyl)methyl]-cyclohexanol with a purity of 98.86% area by HPLC.

### Example 4

### Preparation of 1-[2-dimethylamino-1-(4-methoxyphenyl)ethyl]-cyclohexanol (I)

An autoclave was charged with methanol (200 ml), 1-[Cyano-(4-methoxyphenyl) methyl]-cyclohexanol (II) (25 g, 0.1019 mole), palladium on charcoal (10%, 50% wet) (25 g) and, hydrochloric acid (1-3 mole). While supplying hydrogen gas at 5-25 kg/cm², the mixture was slowly heated to 40° C. and then heated to 40-50° C. for about 7-12 hours with hydrogen pressure 15-20 kg/cm². After completion of the reaction, the mass was cooled to 25-30° C. and the hydrogen pressure was slowly released. The catalyst was filtered off and washed with methanol. Methanol was distilled out at 45-50° C., followed by addition of formic acid (28.14 g, 0.6114 mole), formaldehyde (12.24 g, 0.4076 mole) and water (250 ml). The aqueous reaction mass was heated to reflux for 20-24 h and subsequently was cooled to 20-25° C. The pH of the aqueous mass was adjusted to 9.5-11.5 by adding a solution of 50% sodium hydroxide. The aqueous phase was extracted with ethyl acetate (3×75 ml), followed by washing with water (50 ml) and saturated sodium chloride solution (50 ml) and drying the combined ethyl acetate extracts over anhydrous sodium sulfate. The solvent ethyl acetate was distilled out under vacuum at 45-50° C. and material crystallized out with the addition of n-heptane (50 ml). 21.75 g (77% yield) of a solid 1-[2-Dimethylamino-1-(4-methoxyphenyl)ethyl]-cyclohexanol with a purity of 99% area by HPLC was obtained.

### Example 5

### Preparation of 1-[2-dimethylamino-1-(4-methoxyphenyl)ethyl]-cyclohexanol (I)

An autoclave was charged with methanol (200 ml), 1-[Cyano-(4-methoxyphenyl)methyl]-cyclohexanol (II) (25 g, 0.1019 mole), palladium on charcoal (10%, 50% wet) (25 g), formic acid (3-5 mole equivalent), formaldehyde (2-6 mole equivalent) and water (125 ml). While supplying hydrogen gas at 5-25 kg/cm², the mixture was slowly heated to 40° C. and then heated to 40-50° C. for about 10-24 hours with hydrogen pressure of 10-20 kg/cm². After completion of the reaction, the mass was cooled to 25-30° C. and the hydrogen pressure was slowly released. The catalyst was filtered off and washed with methanol. The solvent methanol was distilled out followed by addition of n-Heptane (250 ml) and charcoal (2 g) at 60-70° C. The pH of the mass was adjusted to 10-11 by adding a solution of 50% sodium hydroxide and stirred for 20-30 minutes at 60-70° C. followed by filtration of the mass at 60-70° C. The filtration bed was washed with hot water (100 ml) followed by washing with hot n-heptane (50 ml). The organic layer was separated and washed with hot water (100 ml). n-Heptane was distilled out at 90-103° C., to obtain venlafaxine free base. The free base was crystallized from n-Heptane (100 ml) first at 50-60° C. and then at 0-5° C. The crystals were filtered, washed with chilled n-Heptane (50 ml) and dried under vacuum at 50-60° C. to yield 21.33 grams (75.5% yield) of venlafaxine free base, i.e., 1-[2-Dimethylamino-1-(4-methoxyphenyl)ethyl]-cyclohexanol, with a purity of 99.6% area by HPLC.

### Example 6

### Preparation of 1-[2-dimethylamino-1-(4-methoxyphenyl)ethyl]-cyclohexanol (I)

An autoclave was charged with methanol (200 ml), 1-[Cyano-(4-methoxyphenyl)methyl]-cyclohexanol (II) (25 g, 0.1019 mole), platinum on charcoal (5%, 15 g), formic acid (3-5 mole equivalent), formaldehyde (2-6 mole equivalent) and water (125 ml). While supplying hydrogen gas at 5-25 kg/cm², the mixture was slowly heated to 40° C. and then heated to 40-50° C. for about 10-24 hours with hydrogen pressure of 10-20 kg/cm². After completion of the reaction, the mass was cooled to 25-30° C. and the hydrogen pressure was slowly released. The catalyst was filtered off and washed with methanol. The solvent methanol was distilled out followed by addition of n-heptane (250 ml) and charcoal (2 g) at 60-70° C. The pH of the mass was adjusted to 10-11 by adding a solution of 50% sodium hydroxide and stirred for 20-30 minutes at 60-70° C. followed by filtration of the mass at 60-70° C. The filtration bed was washed with hot water (100 ml) followed by washing with hot n-heptane (50 ml). The organic layer was separated and washed with hot water (100 ml). n-Heptane was distilled out at 90-103° C., to obtain venlafaxine free base. The free base was crystallized from n-Heptane (100 ml) first at 50-60° C. and then at 0-5° C. The crystals were filtered, washed with chilled n-Heptane (50 ml) and dried under vacuum at 50-60° C. to yield 22 gm (78% yield) of venlafaxine free base, i.e., 1-[2-Dimethylamino-1-(4-methoxyphenyl)ethyl]-cyclohexanol, with a purity of 99% area by HPLC.

### Example 7

### Preparation of 1-[2-amino-1-(4-methoxyphenyl)ethyl]-cyclohexanol (III)

An autoclave was charged with methanol (200 ml), 1-[Cyano-(4-methoxyphenyl)methyl]-cyclohexanol (II) (25 g, 0.1019 mole), palladium on charcoal (10%, 50% wet) (25 g), hydrochloric acid (1-3 mole). While supplying hydrogen gas at 0-20 kg/cm², the mixture was slowly heated to 40° C. and then heated to 40-50° C. for about 7-12 hours with hydrogen pressure 15-20 kg/cm². After completion of the reaction, the mass was cooled to 25-30° C. and the hydrogen pressure was slowly released. The catalyst was filtered off and washed with methanol. 50% solution of sodium hydroxide was added in sufficient quantity to the clear solution to bring pH to 10.5-11.0 and filtered through celite bed. The solvent methanol was distilled out. The residue was extracted with ethyl acetate after diluting it with water. The organic layer was washed with water, brine and distilled off to yield 24.15 g (yield 85.5%) of 1-[2-amino-1-(4-methoxyphenyl)ethyl]-cyclohexanol (III).

### Example 8

### Preparation of 1-[2-dimethylamino-1-(4-methoxyphenyl)ethyl]-cyclohexanol hydrochloride (IV)

A 0.25 L round bottom flask was charged with isopropanol (125 ml), venlafaxine free base (25 g) from examples 2, and 4-6, and 16% isopropanol hydrochloric acid solution (27.5 g) and stirred at temperature of 70-80° C. followed by stirring at a temperature of 5-10° C. for 90-120 minutes. The precipitated solid was filtered, washed with isopropanol (25 ml) and then dried under vacuum at 35-45° C. to yield 21.2 g (75% yield) of a white solid 1-[2-Dimethylamino-1-(4-methoxyphenyl)ethyl]-cyclohexanol hydrochloride with a purity of 99.81% area by HPLC.

### Example 9

### Preparation of 1-[2-amino-1-(4-methoxyphenyl)ethyl]-cyclohexanol hydrochloride (III-a)

A four-necked flask equipped with an addition funnel, thermometer pocket, drying tube and mechanical stirrer, was charged with 1-[2-amino-1-(4-methoxyphenyl)ethyl]-cyclohexanol (III) (20 g, 0.08 mole) in ethyl acetate (40 ml) followed by addition of isopropanol hydrochloric acid (16%) (22 g) and was stirred at 5° C.-10° C. for 40-60 minutes. The solid separated was filtered and washed with ethyl acetate to yield 17.5 g (75%) of 1-[2-amino-1-(4-methoxyphenyl)ethyl]-cyclohexanol hydrochloride (III-a) with a purity of 99.9% area by HPLC.

### Example 10

### Preparation of 1-[2-amino-1-(4-methoxyphenyl)ethyl]-cyclohexanol hydrochloride (III-a)

An autoclave was charged with methanol (200 ml), 1-[Cyano-(4-methoxyphenyl)methyl]-cyclohexanol (II) (25 g, 0.1019 mole), palladium on charcoal (10%, 50% wet) (25 g), and hydrochloric acid (1-3 mole). While supplying hydrogen gas at 0-20 kg/cm², the mixture was slowly heated to 40° C. and then heated to 40° C.-50° C. for about 7-12 hours with hydrogen pressure 15-20 kg/cm². After completion of the reaction, the mass was cooled to 25° C.-30° C. and the hydrogen pressure was slowly released. The catalyst was filtered off and washed with methanol. The pH of the clear solution was adjusted to 10.5-11.0 by the addition of a 50% solution of sodium hydroxide. The solution was then filtered through a celite bed. The solvent methanol was distilled out. The residue was extracted with ethyl acetate after diluting with water. The organic layer was washed with water, brine and the ethyl acetate was distilled out so as to leave behind 2 volumes of the ethyl acetate with the residue. Isopropanol hydrochloric acid (16%) (28 g) was added to the residue and the resulting mixture was stirred at 5° C.-10° C. for 40-60 minutes. The solid separated was filtered and washed with ethyl acetate to yield 18.2 g (72%) of 1-[2-amino-1-(4-methoxyphenyl)ethyl]-cyclohexanol hydrochloride (III-a) with a purity of 99.9% area by HPLC.

### Example 11

### Preparation of 1-[2-Dimethylamino-1-(4-methoxyphenyl)ethyl]-cyclohexanol (I)

A four-necked flask equipped with an addition funnel, thermometer pocket, and mechanical stirrer, was charged with 1-[2-amino-1-(4-methoxyphenyl)ethyl]-cyclohexanol (III) (25.4 g, 0.1019 mol), formic acid 98% (21.53 g, 0.458 mol), p-formaldehyde (8.44 g, 0.2812 mol) and 125 ml water at a temperature of 25° C. to 30° C. The resulting reaction mass was heated to reflux and maintained at reflux for 20-24 hours, and subsequently cooled to 60° C. to 70° C. The pH of the mass was adjusted to 9.5-11.5 by the addition of a solution of 50% sodium hydroxide. Then, heptane (160 ml) and charcoal (5 g) were added, while maintaining the temperature of the mass at 60° C. to 70° C., and the resulting two-phase mixture was stirred for 10-15 minutes. The charcoal was then removed by hot filtration through a celite bed. The filtrate was collected and the layers separated. The organic layer was washed with 25 ml of water at 60° C. to 70° C. and then the solvent was distilled off. The resulting residue was crystallized from n-heptane. The resulting crystals were filtered and dried under vacuum at 40° C. to 50° C. to obtain 18.2-20 g (64.56-70.94% yield) of a white solid of 1-[2-Dimethylamino-1-(4-methoxyphenyl)ethyl]-cyclohexanol (I) with a purity of 99.91% area by HPLC.

### Example 12

### Preparation of 1-[2-Dimethylamino-1-(4-methoxyphenyl)ethyl]-cyclohexanol (I)

A four-necked flask equipped with an addition funnel, thermometer pocket, and mechanical stirrer was charged with 1-[2-amino-1-(4-methoxyphenyl)ethyl]-cyclohexanol hydrochloride (III-a) (31.92 g, 0.1019 mol), 47% sodium hydroxide solution (12.79 g), formic acid 98% (21.53 g, 0.458 mol), p-formaldehyde (8.44 g, 0.2812 mol) and 125 ml water at a temperature of 25° C. to 30° C. The resulting reaction mass was heated to reflux and maintained at reflux for 20-24 hours, and subsequently cooled to 60° C. to 70° C. The pH of the mass was adjusted to 9.5-11.5 by the addition of a solution of 50% sodium hydroxide. Then, heptane (160 ml) and charcoal (5 g) were added, while maintaining the temperature of the mass at 60° C. to 70° C., and the resulting two-phase mixture was stirred for 10-15 minutes. The charcoal was then removed by hot filtration through a celite bed. The filtrate was collected and the layers separated. The organic layer was washed with 25 ml of water at 60° C. to 70° C. and then the solvent was distilled off. The resulting residue was crystallized from n-heptane. The resulting crystals were filtered and dried under vacuum at 40° C. to 50° C. to obtain 18.2-20 g (64.56-70.94% yield) of a white solid of 1-[2-Dimethylamino-1-(4-methoxyphenyl)ethyl]-cyclohexanol (I) with a purity of 99.91% area by HPLC.

We claim:

1. A process for preparing 1-[cyano-(4-methoxyphenyl)methyl]-cyclohexanol of Formula (II) comprising:
a) reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II); and
b) recovering the 1-[cyano-(4-methoxyphenyl)methyl]-cyclohexanol of Formula (II).

2. The process according to claim 1, wherein the alkali aloxide is based on an alkoxide having from 1 to 6 carbon atoms.

3. The process according to claim 1, wherein the alkali alkoxide is sodium ethoxide, sodium methoxide, potassium ethoxide, potassium methoxide, or potassium tert-butoxide.

4. The process according to claim 1, wherein the alkali alkoxide is sodium ethoxide or sodium methoxide.

5. The process according to claim 1, wherein the condensation is performed at a temperature of about −50° C. to about 15° C.

6. The process according to claim 1, wherein the condensation is performed at a temperature of about −10° C. to about 15° C.

7. The process according to claim 1, wherein the condensation is performed at a temperature of about 0° C. to about 5° C.

8. The process according to claim 1, wherein about 1 to about 2 mole equivalents of alkali alkoxide and about 1 to about 3 mole equivalents of cyclohexanone of Formula (VI) are present per mole of p-methoxyphenyl acetonitrile of Formula (V) in step a).

9. The process according to claim 1, wherein about 1.2 to about 1.5 mole equivalents of alkali alkoxide and about 1.5 to about 2 mole equivalents of cyclohexanone of Formula (VI) are present per mole of p-methoxyphenyl acetonitrile of Formula (V) in step a).

10. The process according to claim 1, wherein the 1-[cyano-(4-methoxyphenyl)methyl]-cyclohexanol of Formula (II) is obtained in a yield of at least about 98%.

11. A process for preparing venlafaxine of Formula (I) or a pharmaceutically acceptable salt thereof comprising:
a) reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II);
b) converting the 1-[cyano-(4-methoxyphenyl)methyl]-cyclohexanol of Formula (II) into 1-[2-amino-1-(4-methoxyphenyl)ethyl]-cyclohexanol of Formula (III);
d) converting the 1-[2-amino-1-(4-methoxyphenyl)ethyl]-cyclohexanol of Formula (III) into venlafaxine of Formula (I); and
e) optionally converting the venlafaxine of Formula (I) into a pharmaceutically acceptable salt thereof.

12. A process for preparing 1-[2-amino-1-(4-methoxyphenyl)ethyl]-cyclohexanol of Formula (III) comprising:
a) reacting 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); and
b) recovering the 1-[2-amino-1-(4-methoxyphenyl)ethyl]-cyclohexanol of Formula (III).

13. The process according to claim 12, wherein the hydrogenation catalyst is palladium on charcoal, platinum on charcoal, rhodium on charcoal, or rhodium on alumina.

14. The process according to claim 12, wherein the alcohol is a C₁-C₆ alcohol.

15. The process according to claim 12, wherein the alcohol is methanol, ethanol, isopropanol, n-propanol, n-butanol, isobutanol, or t-butanol.

16. The process according to claim 12, wherein the alcohol is methanol, ethanol, or isopropanol.

17. The process according to claim 12, wherein the inorganic acid is hydrochloric acid, hydrochloric acid from acetyl chloride, ammonium chloride, hydrobromic acid, hydrobromic acid from acetyl bromide, or ammonium bromide.

18. The process according to claim 12, wherein the inorganic acid is present in an amount of 0.5 to 5 mole equivalent per mole equivalent of 1-[cyano-(4-methoxyphenyl)methyl]-cyclohexanol of Formula (II) in step a).

19. The process according to claim 12, wherein the inorganic acid is present in an amount of 1 to 3 mole equivalent per mole equivalent of 1-[cyano-(4-methoxyphenyl)methyl]-cyclohexanol of Formula (II) in step a).

20. The process according to claim 12, wherein the hydrogen is added at a pressure of about 5 to about 25 kg/cm².

21. The process according to claim 12, wherein the hydrogen pressure is added at a pressure of about 10 to about 20 kg/cm².

22. The process according to claim 12, wherein the hydrogen pressure is added at a pressure of about 15 to about 20 kg/cm².

23. The process according to claim 12, wherein a temperature of about 25° C. to about 65° C. is maintained during hydrogenation.

24. The process according to claim 12, wherein a temperature of about 35° C. to about 60° C. is maintained during hydrogenation.

25. The process according to claim 12, wherein a temperature of about 40° C. to about 50° C. is maintained during hydrogenation.

26. A process for preparing 1-[2-amino-1-(4-methoxyphenyl)ethyl]-cyclohexanol of Formula (III) comprising:
a) reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II);
b) reacting the 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III); and
b) recovering the 1-[2-amino-1-(4-methoxyphenyl)ethyl]-cyclohexanol of Formula (III).

27. A process for preparing 1-[2-amino-1-(4-methoxyphenyl)ethyl]-cyclohexanol hydrochloride of Formula (III-a) comprising:
a) combining 1-[2-amino-1-(4-methoxyphenyl)ethyl]-cyclohexanol of Formula (III), a water immiscible solvent and hydrochloric acid to form a mixture; and
b) recovering 1-[2-amino-1-(4-methoxyphenyl)ethyl]-cyclohexanol hydrochloride of Formula (III-a) from the mixture.

28. The process according to claim 27, wherein the 1-[2-amino-1-(4-methoxyphenyl)ethyl]-cyclohexanol hydrochloride of Formula (III-a) is recovered in a purity of greater than about 98% area by HPLC.

29. The process according to claim 27, wherein the 1-[2-amino-1-(4-methoxyphenyl)ethyl]-cyclohexanol hydrochloride of Formula (III-a) is recovered in a purity of about 99.9% area by HPLC.

30. The process according to claim 27, wherein the 1-[2-amino-1-(4-methoxyphenyl)ethyl]-cyclohexanol hydrochloride of Formula (III-a) is recovered in a yield of about 70 to 80%.

31. A process for preparing 1-[2-amino-1-(4-methoxyphenyl)ethyl]-cyclohexanol of Formula (III-a) comprising:
a) reducing 1-[cyano-(4-methoxyphenyl)methyl]-cyclohexanol of Formula (II) with hydrogen and a hydrogenation catalyst, in the presence of at least one inorganic acid in at least one alcohol to form 1-[2-amino-1-(4-methoxyphenyl)ethyl]-cyclohexanol of Formula (III);
b) combining the 1-[2-amino-1-(4-methoxyphenyl)ethyl]-cyclohexanol of Formula (III), a water immiscible solvent and hydrochloric acid to form a mixture; and
c) recovering 1-[2-amino-1-(4-methoxyphenyl)ethyl]-cyclohexanol hydrochloride of Formula (III-a) from the mixture.

32. A process for preparing venlafaxine of Formula (I) or a pharmaceutically acceptable salt thereof comprising:
a) combining 1-[2-amino-1-(4-methoxyphenyl)ethyl]-cyclohexanol of Formula (III) with formaldehyde, formic acid, and water to form a reaction mixture;
b) heating the reaction mixture at reflux for a period of time sufficient to carry out N-methylation of the 1-[2-amino-1-(4-methoxyphenyl)ethyl]-cyclohexanol of Formula (III);
c) cooling the reaction mixture to a temperature of about 25° C. to about 20° C.;
d) adding a base in an amount sufficient to adjust the pH of the reaction mixture to about 9.5 to about 11.5;
e) extracting the aqueous phase with a water immiscible organic solvent to obtain a precipitate of venlafaxine of Formula (I);
f) recovering the precipitated venlafaxine of Formula (I); and
g) optionally converting the venlafaxine of Formula (I) into a pharmaceutically acceptable salt thereof.

33. The process according to claim 32, wherein the base is added in an amount sufficient to adjust the pH of the reaction mixture to about 9.5 to about 10.5.

34. The process according to claim 32, wherein the water immiscible organic solvent is ethyl acetate, n-heptane, toluene, or methylene dichloride.

35. The process according to claim 32, wherein the pharmaceutically acceptable salt is the hydrochloride salt.

36. A process for preparing venlafaxine of Formula (I) or a pharmaceutically acceptable salt thereof comprising:
a) reacting p-methoxyphenyl acetonitrile of Formula (V) with cyclohexanone of Formula (VI) in the presence of at least one alkali alkoxide in at least one of methanol and ethanol to form 1-[cyano-(4-methoxyphenyl)methyl]-cyclohexanol of Formula (II);
b) reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III);
c) combining 1-[2-amino-1-(4-methoxyphenyl)ethyl]-cyclohexanol of Formula (III) with formaldehyde, formic acid, and water to form a reaction mixture;
d) heating the reaction mixture at reflux for a period of time sufficient to carry out N-methylation of the 1-[2-amino-1-(4-methoxyphenyl)ethyl]-cyclohexanol of Formula (III);
e) cooling the reaction mixture to a temperature of about 25° C. to about 20° C.;
f) adding a base in an amount sufficient to adjust the pH of the reaction mixture to about 9.5 to about 11.5;
g) extracting the aqueous phase with a water immiscible organic solvent to obtain a precipitate of venlafaxine of Formula (I);
h) recovering the precipitated venlafaxine of Formula (I); and
i) optionally converting the venlafaxine of Formula (I) into a pharmaceutically acceptable salt thereof.

37. A one-pot process for preparing venlafaxine of Formula (I) or a pharmaceutically acceptable salt thereof comprising:
a) providing 1-[cyano-(4-methoxyphenyl)methyl]-cyclohexanol of Formula (II);
b) reducing the 1-[cyano-(4-methoxyphenyl)methyl]-cyclohexanol of Formula (II) with sodium borohydride and boron trifluoride ethyl ether in at least one aprotic solvent to obtain 1-[2-amino-1-(4-methoxyphenyl)ethyl]-cyclohexanol of Formula (III) in situ;
c) quenching the resulting reaction mass;
d) N-methylating the 1-[2-amino-1-(4-methoxyphenyl)ethyl]-cyclohexanol of Formula (III) with formaldehyde and formic acid to form venlafaxine of Formula (I); and
e) optionally converting the venlafaxine of Formula (I) into a pharmaceutically acceptable salt thereof.

38. The process according to claim 37, wherein about 2 to about 4 mole equivalents of sodium borohydride and about 1 to about 3 mole equivalents of boron trifluoride ethyl ether are present per mole of 1-[cyano-(4-methoxyphenyl)methyl]-cyclohexanol of Formula (II) in step a).

39. The process according to claim 37, wherein the 1-[cyano-(4-methoxyphenyl)methyl]-cyclohexanol of Formula (II), sodium borohydride, and boron trifluoride ethyl ether are combined in a molar ratio of 1:4:3 in step a).

40. The process according to claim 37, wherein the 1-[cyano-(4-methoxyphenyl)methyl]-cyclohexanol of Formula (II), sodium borohydride, and boron trifluoride ethyl ether are combined in a molar ratio of 1:3:2 in step a).

41. The process according to claim 37, wherein the 1-[cyano-(4-methoxyphenyl)methyl]-cyclohexanol of Formula (II), sodium borohydride, and boron trifluoride ethyl ether are combined in a molar ratio of 1:2:1 in step a).

42. The process according to claim 37, wherein the aprotic solvent is tetrahydrofuran, tert-butylmethyl ether, or di-isopropyl methyl ether.

43. The process according to claim 37, wherein the reducing step is carried out at a temperature of about −10° C. to about 45° C.

44. The process according to claim 37, wherein the pharmaceutically acceptable salt is venlafaxine hydrochloride of Formula (IV).

45. The process according to claim 37, wherein the venlafaxine hydrochloride of Formula (IV) is produced in a purity of greater than about 98% area by HPLC.

46. The process according to claim 37, wherein the venlafaxine hydrochloride of Formula (IV) is produced in a purity of greater than about 99% area by HPLC.

47. The process according to claim 37, wherein the venlafaxine hydrochloride of Formula (IV) is produced in a purity of greater than about 99.5% area by HPLC.

48. The process according to claim 37, wherein the venlafaxine hydrochloride of Formula (IV) is produced in a purity of greater than about 99.8% area by HPLC.

49. A one-pot process for preparing venlafaxine of Formula (I) or a pharmaceutically acceptable salt thereof comprising:
a) providing 1-[cyano-(4-methoxyphenyl)methyl]-cyclohexanol of Formula (II);
b) reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III);
c) N-methylating the 1-2(amino-1-(4-methoxyphenyl)ethyl]-cyclohexanol of Formula (III) with formaldehyde and formic acid to form venlafaxine of Formula (I);
d) recovering the venlafaxine of Formula (I) from the reaction mixture; and
e) optionally converting the venlafaxine of Formula (I) into a pharmaceutically acceptable salt thereof.

50. The process according to claim 49, wherein the hydrogenation catalyst is palladium on charcoal, platinum on charcoal, rhodium on charcoal, or rhodium on alumina.

51. The process according to claim 49, wherein the alcohol is a C₁-C₆ alcohol.

52. The process according to claim 49, wherein the alcohol is methanol, ethanol, isopropanol, n-propanol, n-butanol, isobutanol, or t-butanol.

53. The process according to claim 49, wherein the alcohol is methanol, ethanol, or isopropanol.

54. The process according to claim 49, wherein the inorganic acid is hydrochloric acid, hydrochloric acid from acetyl chloride, ammonium chloride, hydrobromic acid, hydrobromic acid from acetyl bromide, or ammonium bromide.

55. The process according to claim 49, wherein the inorganic acid is present in an amount of 0.5 to 5 mole equivalent per mole equivalent of 1-[cyano-(4-methoxyphenyl)methyl]-cyclohexanol of Formula (II) in step a).

56. The process according to claim 49, wherein the inorganic acid is present in an amount of 1 to 3 mole equivalent per mole equivalent of 1-[cyano-(4-methoxyphenyl)methyl]-cyclohexanol of Formula (II) in step a).

57. The process according to claim 49, wherein the hydrogen is added at a pressure of about 5 to about 25 kg/cm².

58. The process according to claim 49, wherein the hydrogen pressure is added at a pressure of about 10 to about 20 kg/cm².

59. The process according to claim 49, wherein the hydrogen pressure is added at a pressure of about 15 to about 20 kg/cm².

60. The process according to claim 49, wherein a temperature of about 25° C. to about 65° C. is maintained during hydrogenation.

61. The process according to claim 49, wherein a temperature of about 35° C. to about 60° C. is maintained during hydrogenation.

62. The process according to claim 49, wherein a temperature of about 40° C. to about 50° C. is maintained during hydrogenation.

63. The process according to claim 49, wherein the pharmaceutically acceptable salt is venlafaxine hydrochloride of Formula (IV).

64. The process according to claim 49, wherein the venlafaxine hydrochloride of Formula (IV) is produced in a purity of greater than about 98% area by HPLC.

65. The process according to claim 49, wherein the venlafaxine hydrochloride of Formula (IV) is produced in a purity of greater than about 99% area by HPLC.

66. The process according to claim 49, wherein the venlafaxine hydrochloride of Formula (IV) is produced in a purity of greater than about 99.5% area by HPLC.

67. The process according to claim 49, wherein the venlafaxine hydrochloride of Formula (IV) is produced in a purity of greater than about 99.8% area by HPLC.

68. A process for preparing venlafaxine of Formula (I) or a pharmaceutically acceptable salt thereof comprising:
a) reacting p-methoxyphenyl acetonitrile of Formula (V) with cyclohexanone of Formula (VI) in the presence of at least one alkali alkoxide in at least one of methanol and ethanol at a temperature of about 10° C. to about 25° C. to form 1-[cyano-(4-methoxyphenyl)methyl]-cyclohexanol of Formula (II);
b) reacting the 1-[cyano-(4-methoxyphenyl)methyl]-cyclohexanol of Formula (II) with hydrogen and a hydrogenation catalyst in the presence of at least one inorganic acid in at least one alcohol to form 1-[2-amino-1-(4-methoxyphenyl)ethyl]-cyclohexanol of Formula (III);
c) N-methylating the 1-[2-amino-1-(4-methoxyphenyl)ethyl]-cyclohexanol of Formula (III) with formaldehyde and formic acid to form venlafaxine of Formula (I); and
d) optionally converting the venlafaxine of Formula (I) into a pharmaceutically acceptable salt thereof.
